(19) United States
(12) Patent Application Publication
(10) Pub. No.: US 2010/0236140 A1
(43) Pub. Date: Sep. 23, 2010

(54) FUEL ADDITIVES WITH IMPROVED MISCIBILITY AND REDUCED TENDENCY TO FORM EMULSIONS

(75) Inventors: Ludwig Voelkel, Limburgerhof (DE); Dietmar Posselt, Heidelberg (DE); Alfred Karl Jung, Rockwood, ME (US); Markus Kummeter, Heddesheim (DE); Gero Nordmann, Charlotte, NC (US); Andrea Misske, Speyer (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/681,788

(22) PCT Filed: Oct. 17, 2008
(86) PCT No.: PCT/EP08/64021

§ 371 Date: Apr. 6, 2010

(30) Foreign Application Priority Data
Oct. 19, 2007 (EP) 07118841.1

(51) Int. Cl. C10L 10/00; C07C 215/02

(52) U.S. Cl. 44/433; 564/503

(57) ABSTRACT

The present invention relates to novel fuel additives obtainable by reacting carboxylic acids and alkanol amines under specific conditions. Said additives show an improved performance in fuels, like gasoline. The invention also relates to methods of preparing the same; additive packages containing said additives; and methods of improving the storage stability of additive packages comprising a detergent additive in an organic solvent.

### FIELD OF THE INVENTION

The present invention relates to novel fuel additives obtainable by reacting carboxylic acids and alkanol amines under specific conditions. Said additives show an improved performance in fuels, like gasoline. The invention also relates to methods of preparing the same; additive packages containing said additives; and methods of improving the storage stability of additive packages comprising a detergent additive in an organic solvent.

### BACKGROUND OF THE INVENTION

Reaction products of fatty acid derivatives and alkanol mono- or polyamines are known to be useful additives for application in gasoline and diesel.

Chapter 7: Organic Friction Modifiers, Lubricant Additives: Chemistry and Applications; Leslie R. Rudnick, CRC 2003, ISBN 0824708571. Kenbeek and Buenemann explain that non-acetic organic friction modifiers are preferably long straight-chain molecules with small polar heads. They are described to form adsorption layers on the surface where multiple molecules are adsorbed by hydrogen bonding and Debye orientation forces. Van der Waals forces cause the molecules to align themselves such that they form multimolecular clusters that are parallel to each other. Examples of organic friction modifiers are oleylamide and glycerol mono-oleate (GMO).

EP 1 295 933 describes deposit control additives for direct injected engines available by reaction of monocarboxylic acids and polyamines. Most preferred is a molar ratio of 1 to 1.5 moles of monocarboxylic acid and 1 mole of polyamine. Specific preferred examples are the reaction products of equimolar amounts of tallow fatty acid or oleic acid and AEAE. According to the general procedure disclosed therein the reaction is performed at reflux temperature which is in the range of 150 to 175° C. There is no suggestion made in said document with respect to choosing the reaction conditions (molar ratio and/or reaction temperature) such that polysubstituted alkanolamines are preferentially formed. In particular, it is not suggested to control the kinetics of the reaction by selecting a suitable temperature profile.

EP 1 435 386 describes fatty acid alkanol amides, which improve the acceleration properties of internal combustion engines. This document describes alkanol monoamides obtainable by reaction of 1 mole of fatty acid or it's esters and 1 mole of an alkanol monoamine.

EP1 272 594 describes the use of friction modifiers, which are the reaction products of certain natural or synthetic carboxylic acid glyceryl esters and alkanol amines in combination with a detergent additive in gasoline for improving the delivery of the friction modifier to the lubricant of the engine. The reaction of preparing the friction modifier is performed without applying a specific temperature profile. The specific selection of a significant molar excess of the alkanol amine is neither suggested nor exemplified. Similar friction modifiers are disclosed on WO 2007/053787 where it is suggested to use the same in combination with a solvent, an alcohol and certain compatibilizer to form fuel additive concentrates remaining fluid at −8° C. or below.

Even if these additives provide good performance, they have significant disadvantages due to their polar structure.

Most of such components stabilize emulsions of hydrocarbon fuel and water. Such emulsions can cause severe damage in modern cars; additive suppliers therefore need to compensate this effect by adding so called dehazers.

Furthermore most of such 1:1 adducts of a carboxylic acid moiety and an alkanol amine show a strong tendency to form multi-molecular clusters, which results in incompatibility with typical detergent additives such as PIB monoamines, PIB Mannich amines or PIB succinimides. Blends of such fatty acid amides with PIB-based products therefore require expensive solvents such as hydrophobic alcohols or comparable solubilizer.

Even if there are technical solutions to overcome these problems, they will at least unfavourably increase costs so that some of these additives are not economically advantageous.

The problem to be solved by the present invention, therefore, was to develop additives, which show better solubility and compatibility as well as milder emulsion behaviour than conventional reaction products of fatty acids and alkanol amines, while, preferably, maintaining a similar additive performance profile. In particular, the additives of the present invention should improve the storage stability of additive packages, in particular at temperatures below 0° C., and should improve the phase separation of fuel/water emulsions so that less or no dehazer is required for preparing the fuel.

### SUMMARY OF THE INVENTION

Surprisingly is was found that the conversion products of carboxylic acids and alkanol amines obtained under specific reaction conditions and which result in the formation of specific complex reaction mixtures comprising substantial proportions of low polarity constituents still have sufficient additive performance in the fuel, in particular gasoline. An addition, due to their lower polarity they are better compatible with other additive compounds and need no or less dehazer to compensate emulsion effects.

### DETAILED DESCRIPTION OF THE INVENTION

### A) Preferred Embodiments

A first embodiment of the invention relates to a reaction product, obtainable by reacting, preferably in a thermal condensation reaction, a carboxylic acid (or carboxylate) compound of formula I

R¹COOR² (I)

in which

R¹ is an aliphatic C₁-C₃₀-hydrocarbon radical;

R² is hydrogen or alkyl, mono- or polyhydroxyalkyl, or ammonium,

with an alkanol amine of the formula II

NHR³R⁴ (II)

wherein R³ and R⁴ are independently selected from hydrogen atoms and linear or branched-chain hydrocarbon groups, the carbon chain of which optionally being interrupted by one or more —NH— groups, and which optionally has at least one hydroxyl group attached to a carbon atom, with the proviso that R³ and R⁴ are not both hydrogen atoms and that at least one of said residues R³ and R⁴ carries at least one hydroxyalkyl group,

in a molar ratio of the carboxyl groups (—COO—) of the carboxylic acid of formula I to the molar sum of OH and NH groups of the alkanol amine of formula II in a range and under reaction conditions supporting the formation of a reaction product comprising poly-substituted alkanol amine derivatives.

Preferably, said polysubstituted (as for example polycarbonylated) alkanol amine derivatives are comprised in said reaction product in a proportion of more than 20 wt.-%, preferably more than 40 wt.-%, and in particular more than 60 wt.-%, based on the total weight of the reaction product.

On the other hand 1:1 adducts are present in a total amount of 20 wt.-% or less, more preferred at 15 wt.-% or less and most preferred at a level of 10 wt.-% or less, like about 0,1 to about 10 or about 1 to about 8 or about 1,5 to about 5, about 2 to about 4 wt.-%, based on the total weight of the reaction product.

According to a further preferred embodiment the reaction product of the invention is obtained by a process, wherein the molar ratio of the carboxyl groups of the carboxylic acid of formula I to the molar sum of OH and NH groups of the alkanol amine of formula II is in the range of about 1.8:3 to 3:3, in particular 1.9:3 to 2.5:3.

Preferably said reaction is performed by
a) heating the carboxylic acid compound(s) of formula I (in substance or dissolved/ or dispersed in a suitable liquid which does not disturb the reaction) to a first temperature in a first temperature range, allowing the preferential reaction of the acid with amine group(s) of the alkanol amine;
b) adding thereto the alkanol amine compound(s) of formula II (in substance or dissolved or dispersed in a suitable liquid which does not disturb the reaction) under controlled conditions in order to avoid an increase of the temperature above said first temperature range;
c) reacting the compounds by maintaining the temperature in said first range;
d) and increasing the first temperature of the reaction mixture to a second temperature in a second temperature range allowing further condensation of residual free carboxylic acid molecules with any reactive group in the reaction mixture, preferably until the amount of water condensate is at least equal to the theoretical amount of reaction water.

Preferably, the first temperature in step a), b) and/or c) is kept in the range of about 100 to about 155° C., as for example about 110 to about 140° C., or about 120 to about 135° C.

Preferably, the second temperature in step d) is kept in the range of 160 to 210° C., as for example about 170 to about 200° C., or about 175 to about 190° C.

In a particularly preferred embodiment the reaction product the additive is obtained by reacting a carboxylic acid compound with an alkanol amine of formula II, wherein R³ and R⁴ independently of each other represent hydrogen or a residue of the formula III

—[(CH₂)ₓNH]_{y}(CH₂)_{z}R⁵ (III)
wherein
x and z are independently from each other integers from 1 to 6, preferably 1, 2, or 3,
y is 0 or an integer of 1 to 3, preferably 0 or 1, and
R⁵ is hydroxyl or a residue of the formula IV

—NH(CH₂)_{z}OH (IV)
wherein z is as defined above; with the proviso that R³ and R⁴ are not both hydrogen atoms.

In a further particularly preferred embodiment the reaction product is obtained from a compound of formula I, which is selected from C₂-C₃₁— or C₈-C₃₁— or C₈-C₃₀— or C₁₀-C₂₂-carboxylic acids and alkyl esters thereof.

Preferably the compound of formula II is selected from polyamino alkanols, wherein one of the residues R³ and R⁴ is hydrogen and the other is a residue of the formula III, wherein x is 2 or 3, y is 0 or 1, z is 2 or 3 and R⁵ is hydroxyl or a residue of the formula IV.

According to another embodiment of the invention additive packages are provided, comprising in a suitable organic solvent at least one detergent additive and at least one reaction product as defined above.

According to another embodiment of the invention a method of improving the storage stability of additive packages wherein the additive package comprises at least one detergent additive in an organic solvent, which method comprises adding to said package at least one reaction product of the invention. In particular, said detergent additive is selected from as polyalkene monoamines, polyalkene Mannich amines or polyalkene succinimides.

### B) General Definitions

A “reaction product” as used herein means the product of a specific reaction of at least one carboxylic acid compound or a carboxylic acid compound containing first reactant, and at least one alkanol amine or an alkanol amine containing second reactant as explained in more detail below. The reaction product is complex in nature, i.e. consists of a complex mixture of constituents, the profile of which being substantially predetermined by the reaction conditions of said conversion. The reaction product is, as such, a suitable additive for fuels and normally need not be further purified prior to use. The product may, however, be concentrated (if necessary) in order to remove residual solvent or low molecular constituents, like water or non-reacted reactants, if any.

The term “carboxylate compound” refers to any compound of formula I as defined d above.

The term “aliphatic C₁-C₃₀-hydrocarbon radical” denotes an acyclic radical which is composed substantially of carbon atoms and hydrogen atoms and comprises from 1 to 30, as for example 8 to 30 carbon atoms. The hydrocarbon radical is preferably an alkyl, alkenyl, alkadienyl, alkatrienyl or polyenyl radical. Those skilled in the art will appreciate thr minimum numbers of carbon atoms that need to be present in hydrocarbon radicals of various degree of unsaturation.

An alkyl radical comprises C₁-C₈-alkyl radicals which are linear or branched radicals having from 1 to 8 carbon atoms. Examples thereof are the C₁-C₄-alkyl radicals methyl, ethyl, n-propyl, isopropyl, n-butyl, 2-butyl, isobutyl or tert-butyl, and additionally pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl and their constitutional isomers such as 2-ethylhexyl; or C₈-C₃₀-alkyl radicals which are linear or branched radicals having from 8 to 30 carbon atoms. Examples thereof are octyl, nonyl, decyl, undecyl, dodecyl, tridecyl, tetradecyl, pentadecyl, hexadecyl, heptadecyl, octadecyl, nonadecyl, eicosyl, hencosyl, docosyl, tricosyl, tetracosyl, pentacosyl, hexacosyl, heptacosyl, octacosyl, nonacosyl, squalyl, their constitutional isomers, higher homologs and constitutional isomers thereof.

Alkenyl comprises C₂-C₈-alkenyl radicals which are monounsaturated linear or branched hydrocarbon radicals having from 2 to 8 carbon atoms, as for example ethenyl, 1- or 2-propenyl, 1-, 2- and 3-butenyl, 2-methylpropen-3-yl, 2-methylpropen-1-yl, 1-, 2-, 3- and 4-pentenyl, 1-, 2-, 3-, 4- and 5-hexenyl, 1-, 2-, 3-, 4-, 5- and 6-heptenyl 1-, 2-, 3-, 4-, 5-, 6- and 7-octenyl and also their constitutional isomers; C₈-C₃₀-Alkenyl is a monounsaturated linear or branched hydrocarbon radical having from 8 to 30 carbon atoms. Examples thereof are octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, hencosenyl, docosenyl, tricosenyl, tetracosenyl, pentacosenyl, hexacosenyl, heptacosenyl, octacosenyl, nonacosenyl, squalenyl, their constitutional isomers, higher homologs and constitutional isomers thereof.

Alkandienyl radicals comprise C₄-C₈-alkadienyl radicals which are diunsaturated linear or branched hydrocarbon radical having from 4 to 8 carbon atoms, as for example butadienyl, pentadienyl, hexadienyl, heptadienyl or octadienyl and their constitutional isomers; or C₈-C₃₀-alkadienyl radicals which are diunsaturated linear or branched hydrocarbon radicals having from 8 to 30 carbon atoms. Examples thereof are octadienyl, nonadienyl, decadienyl, undecadienyl, dodecadienyl, tridecadienyl, tetradecadienyl, pentadecadienyl, hexadecadienyl, heptadecadienyl, octadecadienyl, nonadecadienyl, eicosadienyl, hencosadienyl, docosadienyl, tricosadienyl, tetracosadienyl, pentacosadienyl, hexacosadienyl, heptacosadienyl, octacosadienyl, nonacosadienyl, squaladienyl, their constitutional isomers, higher homologs and constitutional isomers thereof. The olefinic double bonds may be present in conjugated or isolated form

Alkantrienyl radicals comprise C₆-C₈-alkatrienyl radicals which are tri-unsaturated linear or branched hydrocarbon radical having from 6 to 8 carbon atoms, as for example hexatrienyl, heptatrienly or octatrienyl; or C₈-C₃₀-alkatrienyl radicals, which are triunsaturated linear or branched hydrocarbon radicals having from 8 to 30 carbon atoms. Examples thereof are octatrienyl, nonatrienyl, decatrienyl, undecatrienyl, dodecatrienyl, tridecatrienyl, tetradecatrienyl, pentadecatrienyl, hexadecatrienyl, heptadecatrienyl, octadecatrienyl, nonadecatrienyl, eicosatrienyl, hencosatrienyl, docosatrienyl, tricosatrienyl, tetracosatrienyl, pentacosatrienyl, hexacosatrienyl, heptacosatrienyl, octacosatrienyl, nonacosatrienyl, squalatrienyl, their constitutional isomers, higher homologs and constitutional isomers thereof. The olefinic double bonds may be present in conjugated or isolated form.

Polyenyl radicals are in particular C₈-C₃₀-polyenyl radicals which are generally unsaturated linear or branched aliphatic hydrocarbon radicals having from 8 to 30 carbon atoms and four, five, six or more olefinic nonvicinal double bonds Examples thereof are the higher unsaturated analogs of the above C₈-C₃₀-alkadi- and trienyl residues.

Unless otherwise stated the term “alkyl”, as for example in the context of residue R² refers to C₁-C₈-alkyl as defined above.

The term “mono- or polyhydroxyalkyl” refers to C₁-C₈-hydroxyalkyl which is a linear or branched alkyl radical having from 1 to 8, in particular from 1 to 4 carbon atoms, in which at least one hydrogen atom, for example 1, 2, 3, or 4 of the hydrogen atoms, is/are replaced by a hydroxyl group. Examples thereof are, hydroxymethyl, 2-hydroxy-1-ethyl, 2- and 3-hydroxy-1-propyl, 2-, 3- and 4-hydroxy-1-butyl, 2-, 3-, 4- and 5-hydroxy-1-pentyl, 2-, 3-, 4-, 5- and 6-hydroxy-1-hexyl, 2-, 3-, 4-, 5-, 6- and 7-hydroxy-1-heptyl, 2-, 3-, 4-, 5-, 6-, 7- and 8-hydroxy-i-octyl, 2,3-dihydroxy-1-propyl and their constitutional isomers. If R² represents a polyhydroxyalkyl residue, said hydroxy groups are, preferably, not further esterified. In particular, compounds of formular I do not comprise polyol polyester, as for example tri-glycerides.

The term “hydroxyalkyl” refers to C₁-C₈-hydroxyalkyl which is a linear or branched alkyl radical having from 1 to 8, in particular from 1 to 4 carbon atoms, in which one hydrogen atom is replaced by a hydroxyl group. Suitable examples are stated above.

A “linear or branched-chain hydrocarbon group, the carbon chain of which optionally being interrupted by one or more —NH— groups optionally carrying at least one hydroxyalkyl group”, comprises a terminal hydroxyalkyl group which is a mono- or polyhydroxyalkyl group as defined above, and comprises optionally at least one C₁-C₆-alkylene group, optionally substituted by 1 or more, like 1, 2, or 3 hydroxyl groups, whereby two or more of said alkylene groups being linked together by a —NH-group.

“C₁-C₆-Alkylene” is a linear or branched bridging hydrocarbon group having 2, 3, 4, 5 or 6 carbon atoms, such as 1,2-ethylene, 1,2- and 1,3-propylene, 1,2-, 1,3-, 2,3- and 1,4-butylene, 2,2-dimethyl-1,2-ethylene, 1,1-dimethyl-1,2-ethylene, 1,5-pentylene, 1,6-hexylene and constitutional isomers thereof.

A “polysubstituted” or “polycarbonylated” alkanol amine derivative is derived from an polyfunctional alkanol amine, as for example an alkanol polyamine, wherein more than one functional groups (—NH— or —OH groups) of which, being substituted by a carbonyl residue of the formula —CO(hydrocarbyl), wherein hydrocarbyl has the same meanings as an “aliphatic C₁-C₃₀-hydrocarbon radical” as already defined above. In particular said substituents may be derived from same or different C₁₀-C₂₂-carboxylic acids. The term “polysubstituted” encompasses di-, tri-, tetra and higher substituted alkanol amine derivatives.

A “C₂-C₃₁-carboxylic acid” represents a straight-chain or branched, saturated or mono- or poly-unsaturated C₁₋₃₀-hydrocarbyl residue. In particular, said residue is a straight-chain mono- or poly-unsaturated hydrocarbyl residue or a mixture of such residues with an average length of 1-30, 1-29, preferably 5-25 carbon atoms. Particularly preferred residues are:
residues derived from saturated, straight-chain carboxylic acids: CH₃—, C₂H₅—; C₃H₇—; C₄H₉—; C₅H₁₁—; C₆H₁₃—; C₇H₁₅—, C₈H₁₇—; C₉H₁₉—; C₁₀H₂₁—; C₁₁H₂₃—; C₁₂H₂₅—; C₁₃H₂₇—; C₁₄H₂₉—; C₁₅H₃₁—; C₁₆H₃₃—; C₁₇H₃₅—; C₁₈H₃₇—; C₁₉H₃₉—; C₂₀H₄₁—; C₂₁H₄₃—; C₂₃H₄₇—; C₂₄H₄₉; C₂₅H₅₁—; C₂₉H₅₉—; C₃₀H₆₁;
residues derived from saturated, branched carboxylic acids: iso-C₃H₇—; isoC₄H₉—; iso-C₁₈H₃₇—;
residues derived from mono-unsaturated, straight-chain carboxylic acids: C₂H₃—; C₃H₅—; C₁₅H₂₉—; C₁₇H₃₃—; C₂₁H₄₁—;
residues derived from two-fold unsaturated, straight-chain carboxylic acids: C₅H₇—; C₁₇H₃₁—;
residues derived from three-fold unsaturated, straight-chain carboxylic acids: C₁₇H₂₉—;
residues derived from four-fold unsaturated, straight-chain carboxylic acids: C₁₉H₃₁—;
residues derived from five-fold unsaturated, straight-chain carboxylic acids: C₂₁H₃₃—.

Said hydrocarbyl residue may also be derived from fatty acid mixtures as obtained from naturally occurring oils and fats. Non-limiting examples thereof are olive oil, palm oil, palm cernel oil, peanut oil, rapeseed oil, safflower oil, sesame oil, sunflower oil, soy bean oil, to beef tallow oil, lard oil, castor oil, cottonseed oil, corn oil, soybean oil, whale oil, and coconut oil. As examples of suitable fatty acids there may be mentioned monocarboxylic acids such as capric, lauric, myristic, palmitic, stearic, behenic, oleic, petroselinic, elaidic, palmitoleic, linoleic, linolenic and erucic acid.

The term “alkanol amines” has to be understood broadly. It comprises monoalkanolamines, dialkanolamines, and so forth. The alkanolamine can possess one or more additional O and/or N functionalities in addition to the one amino group. and at least one hydroxy group. Suitable alkanolamines include monoethanolamine, diethanolamine, propanolamine, isopropanolamine, dipropanolamine, di-isopropanolamine, butanolamines, and polyaminoalkanols like aminoethylaminoethanols, e.g., 2-(2-aminoethylamino)ethanol (AEAE)

Alkanol amines are, for example, compounds of formula II wherein at least one of the residues R³ and R⁴ represents —[(CH₂)ₓNH]_{y}(CH₂)_{z}R⁵ wherein R⁵ is hydroxyl or NH(CH₂)_{z}OH. Suitable examples of groups of the formula —[(CH₂)ₓNH]_{y}(CH₂)_{z}— are C₂H₄—NHₙC₂H₄, (CH₂)₃—NHₙ(CH₂)₃—, CH₂—CH(CH₃)—NHₙCH₂—CH(CH₃)—, CH(CH₃)—CH₂—NHₙCH(CH₃)—CH₂—, (CH₂)₄—NHₙ(CH₂)₄—, wherein n is 0, 1 or 2.

In one particular group one of R³ and R⁴ represents H, and in the other R⁵ is hydroxyl and —[(CH₂)ₓNH]_{y}(CH₂)_{z} is selected from C₂H₄—NHₙC₂H₄, (CH₂)₃—NHₙ(CH₂)₃—, CH₂—CH(CH₃)—NHₙCH₂—CH(CH₃)—, CH(CH₃)—CH₂—NHₙCH(CH₃)—CH₂—, CH₂)₄—NHₙ(CH₂)₄—, while n is 1 or 2.

### C) Examples of Reaction Products

In a non-limiting example of the present invention the reaction product may represent a complex product mixture, which is characterized by a high proportion of polysubstituted, i.e. at least two-fold substituted, alkanol polyamines (or polyaminoalkanols). In particular, the reaction mixture is characterized by a high proportion of constituents, which are selectively carbonylated at primary and/or secondary amino groups.

Preferably, such reaction products are obtainable by reaction of an alkanol amine selected from the above-identified group of specific alkanol amines with a carboxylic acid containing reagent under conditions defined herein.

Exemplified by AEAE as reactant of formula II the reaction product formed (when a molar excess of a fatty acid is used) may contain main constituents A, B and C (as depicted below), which are: the main diamide product (A), optionally in admixture with the corresponding (analytically difficult to distinguish) monoamidoester, each of which carrying two carbonyl residues; the fully substituted diamidoester (B) carrying three carbonyl groups; and the monoamide (C). The reaction mixture may also contain minor amounts of unreacted oleic acid (D) (1-5%) and AEAE (<0.1%) as well as significant amounts (10-20%) of unidentified by-products (it is presumed that i.a. pyrazidins, imidazolins and ethers are produced). The kinetically controlled first step of the reaction, performed at about 130° C., favors the formation of the main component, in particular the diamide (A), while the less specific reaction conditions in the second reaction step at about 180° C. result in the formation of the diamidoester (B).

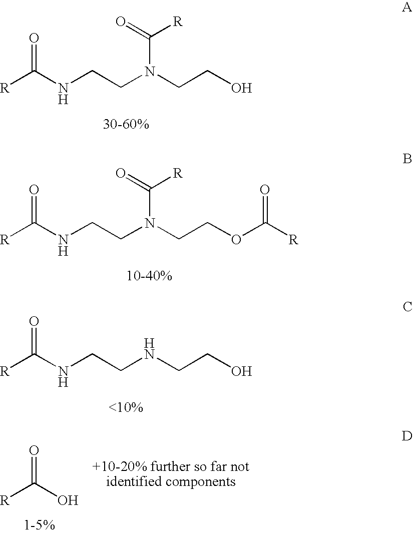

It is well understood by a skilled reader that the specific conditions exemplified herein may be changed without changing the general teaching of the present invention. For example, it is possible to change the order of adding reactants to the reaction mixture, to pre-heat the reactants, if necessary, to add one or more solvents which may be removed after the end of the reaction. In addition it may be possible to remove, if necessary the water as formed during the course of the condensation reaction. Suitable catalysts well-known in the art might also be used.

As suitable solvents there may be used any solvent, which does not negatively affect the conversion reaction, and, optionally which is compatible with the other constituents of an additive package or the fuel to which the additive of the invention has to be added, so that it is not necessary to remove the solvent prior to use. As examples there may be mentioned toluene, xylene or any other aromatic solvent; dioxane, dialkyl glycol and dialkyl oligo glycols.

### D) Further Additive Components

The reaction products of the present invention may be added to the fuel as friction modifier, lubricity additive, detergent or deposit control additive, acceleration improver, or corrosion inhibitor

The reaction products of the invention may be added to the fuels individually or in a mixture with further effective additive components (co-additives) as exemplified in ore detail below.

### D1) Detergent Additives

Examples include additives comprising detergent action (hereinafter referred to as detergent additives). This detergent additive has at least one hydrophobic hydrocarbon radical having a number-average molecular weight (Mn) of from 85 to 20 000 and at least one polar moiety selected from:

(a) mono- or polyamino groups having up to 6 nitrogen atoms, of which at least one nitrogen atom has basic properties;
(b) nitro groups, if appropriate in combination with hydroxyl groups;
(c) hydroxyl groups in combination with mono- or polyamino groups, in which at least one nitrogen atom has basic properties;
(d) carboxyl groups or their alkali metal or their alkaline earth metal salts;
(e) sulfonic acid groups or their alkali metal or alkaline earth metal salts;
(f) polyoxy-C₂- to —C₄-alkylene groups which are terminated by hydroxyl groups, mono- or polyamino groups, in which at least one nitrogen atom has basic properties, or by carbamate groups;
(g) carboxylic ester groups;
(h) moieties derived from succinic anhydride and having hydroxyl and/or amino and/or amido and/or imido groups; and/or
(i) moieties obtained by Mannich reaction of substituted phenols with aldehydes and mono- or polyamines.

The hydrophobic hydrocarbon radical in the above detergent additives, which ensures the adequate solubility in the fuel, has a number-average molecular weight (Mn) of from 85 to 20 000, especially from 113 to 10 000, in particular from 300 to 5000. Typical hydrophobic hydrocarbon radicals, especially in conjunction with the polar moieties (a), (c), (h) and (i), include the polypropenyl, polybutenyl and polyisobutenyl radical each having Mn=from 300 to 5000, especially from 500 to 2500, in particular from 700 to 2300.

Non-limiting examples of the above groups of detergent additives include the following:

Additives comprising mono- or polyamino groups (a) are preferably polyalkenemono- or polyalkenepolyamines based on polypropene or conventional (i.e. having predominantly internal double bonds) polybutene or polyisobutene having Mn=from 300 to 5000. When polybutene or polyisobutene having predominantly internal double bonds (usually in the beta and gamma position) are used as starting materials in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be, for example, ammonia, monoamines or polyamines, such as dimethylaminopropylamine, ethylenediamine, diethylenetriamine, triethylenetetramine or tetraethylenepentamine. Corresponding additives based on polypropene are described in particular in WO-A-94/24231.

Further preferred additives comprising monoamino groups (a) are the hydrogenation products of the reaction products of polyisobutenes having an average degree of polymerization P=from 5 to 100 with nitrogen oxides or mixtures of nitrogen oxides and oxygen, as described in particular in WO-A-97/03946.

Further preferred additives comprising monoamino groups (a) are the compounds obtainable from polyisobutene epoxides by reaction with amines and subsequent dehydration and reduction of the amino alcohols, as described in particular in DE-A-196 20 262.

Additives comprising nitro groups (b), if appropriate in combination with hydroxyl groups, are preferably reaction products of polyisobutenes having an average degree of polymerization P=from 5 to 100 or from 10 to 100 with nitrogen oxides or mixtures of nitrogen oxides and oxygen, as described in particular in WO-A-96/03367 and WO-A-96/03479. These reaction products are generally mixtures of pure nitropolyisobutenes (e.g. alpha,beta-dinitropolyisobutene) and mixed hydroxynitropolyisobutenes (e.g. alpha-nitro-beta-hydroxypolyisobutene).

Additives comprising hydroxyl groups in combination with mono- or polyamino groups (c) are in particular reaction products of polyisobutene epoxides obtainable from polyisobutene having preferably predominantly terminal double bonds and Mn=from 300 to 5000, with ammonia or mono- or polyamines, as described in particular in EP-A-476 485.

Additives comprising carboxyl groups or their alkali metal or alkaline earth metal salts (d) are preferably copolymers of C₂-C₄₀-olefins with maleic anhydride which have a total molar mass of from 500 to 20 000 and of whose carboxyl groups some or all have been converted to the alkali metal or alkaline earth metal salts and any remainder of the carboxyl groups has been reacted with alcohols or amines. Such additives are disclosed in particular by EP-A-307 815. Such additives serve mainly to prevent valve seat wear and can, as described in WO-A-87/01126, advantageously be used in combination with customary fuel detergents such as poly(iso)buteneamines or polyetheramines.

Additives comprising sulfonic acid groups or their alkali metal or alkaline earth metal salts (e) are preferably alkali metal or alkaline earth metal salts of an alkyl sulfosuccinate, as described in particular in EP-A-639 632. Such additives serve mainly to prevent valve seat wear and can be used advantageously in combination with customary fuel detergents such as poly(iso)buteneamines or polyetheramines.

Additives comprising polyoxy-C₂-C₄-alkylene moieties (f) are preferably polyethers or polyetheramines which are obtainable by reaction of C₂- to C₆₀-alkanols, C₆- to C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl group or amino group and, in the case of the polyetheramines, by subsequent reductive amination with ammonia, monoamines or polyamines. Such products are described in particular in EP-A-310 875, EP-A-356 725, EP-A-700 985 and U.S. Pat. No. 4,877,416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (g) are preferably esters of mono-, di- or tricarboxylic acids with long-chain alkanols or polyols, in particular those having a minimum viscosity of 2 mm²/s at 100° C., as described in particular in DE-A-38 38 918. The mono-, di- or tricarboxylic acids used may be aliphatic or aromatic acids, and particularly suitable ester alcohols or ester polyols are long-chain representatives having, for example, from 6 to 24 carbon atoms. Typical representatives of the esters are adipates, phthalates, isophthalates, terephthalates and trimellitates of isooctanol, of isononanol, of isodecanol and of isotridecanol. Such products also have carrier oil properties.

Additives comprising moieties derived from succinic anhydride and having hydroxyl and/or amino and/or amido and/or imido groups (h) are preferably corresponding derivatives of polyisobutenylsuccinic anhydride which are obtainable by reacting conventional or highly reactive polyisobutene having Mn=from 300 to 5000 with maleic anhydride by a thermal route or via the chlorinated polyisobutene. Particular interest attaches to derivatives with aliphatic polyamines such as ethylenediamine, diethylenetriamine, triethylenetetramine or tetraethylenepentamine. Such gasoline fuel additives are described in particular in U.S. Pat. No. 4,849,572.

Additives comprising moieties obtained by Mannich reaction of substituted phenols with aldehydes and mono- or polyamines (i) are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or highly reactive polyisobutene having Mn=from 300 to 5000. Such “polyisobutene-Mannich bases” are described in particular in EP-A-831 141.

For a more precise definition of the gasoline fuel additives detailed individually, reference is explicitly made here to the disclosures of the abovementioned prior art documents, incorporated herewith by reference.

### D2) Carrier Oils

The additive formulations according to the invention may additionally be combined with still further customary components and additives. Mention should be made here primerily of carrier oils.

Suitable mineral carrier oils are the fractions obtained in crude oil processing, such as brightstock or base oils having viscosities, for example, from the SN 500-2000 class; and also aromatic hydrocarbons, paraffinic hydrocarbons and alkoxyalkanols. Likewise useful is a fraction which is obtained in the refining of mineral oil and is known as “hydrocrack oil” (vacuum distillate cut having a boiling range of from about 360 to 500° C., obtainable from natural mineral oil which has been catalytically hydrogenated under high pressure and isomerized and also deparaffinized). Likewise suitable are mixtures of abovementioned mineral carrier oils.

Examples of synthetic carrier oils which are useful in accordance with the invention are selected from: polyolefins (poly-alpha-olefins or poly(internal olefin)s), (poly)esters, (poly)alkoxylates, polyethers, aliphatic polyether amines, alkylphenol-started polyethers, alkylphenol-started polyether amines and carboxylic esters of long-chain alkanols.

Examples of suitable polyolefins are olefin polymers having Mn=from 400 to 1800, in particular based on polybutene or polyisobutene (hydrogenated or nonhydrogenated).

Examples of suitable polyethers or polyetheramines are preferably compounds comprising polyoxy-C₂-C₄-alkylene moieties which are obtainable by reacting C₂-C₆₀-alkanols, C₆-C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl group or amino group, and, in the case of the polyether amines, by subsequent reductive amination with ammonia, monoamines or polyamines. Such products are described in particular in EP-A-310 875, EP-A-356 725, EP-A-700 985 and U.S. Pat. No. 4,877,416. For example, the polyether amines used may be poly-C₂-C₆-alkylene oxide amines or functional derivatives thereof. Typical examples thereof are tridecanol butoxylates or isotridecanol butoxylates, isononylphenol butoxylates and also polyisobutenol butoxylates and propoxylates, and also the corresponding reaction products with ammonia.

Examples of carboxylic esters of long-chain alkanols are in particular esters of mono-, di- or tricarboxylic acids with long-chain alkanols or polyols, as described in particular in DE-A-38 38 918. The mono-, di- or tricarboxylic acids used may be aliphatic or aromatic acids; suitable ester alcohols or polyols are in particular long-chain representatives having, for example, from 6 to 24 carbon atoms. Typical representatives of the esters are adipates, phthalates, isophthalates, terephthalates and trimellitates of isooctanol, isononanol, isodecanol and isotridecanol, for example di-(n- or isotridecyl) phthalate.

Further suitable carrier oil systems are described, for example, in DE-A-38 26 608, DE-A-41 42 241, DE-A-43 09 074, EP-A-0 452 328 and EP-A-0 548 617, which are explicitly incorporated herein by way of reference.

Examples of particularly suitable synthetic carrier oils are alcohol-started polyethers having from about 5 to 35, for example from about 5 to 30, C₃-C₆-alkylene oxide units, for example selected from propylene oxide, n-butylene oxide and isobutylene oxide units, or mixtures thereof. Nonlimiting examples of suitable starter alcohols are long-chain alkanols or phenols substituted by long-chain alkyl in which the long-chain alkyl radical is in particular a straight-chain or branched C₆-C₁₈-alkyl radical. Preferred examples include tridecanol and nonylphenol.

Further suitable synthetic carrier oils are alkoxylated alkylphenols, as described in DE-A-10 102 913.6.

### D3) Solvents and Co-Solvents

As examples of suitable solvent: any type of hydrocarbon solvent may be mentioned, e.g. kerosene, heavy aromatic solvent (“solvent naphta heavy”, “Solvesso 150”), xylene, paraffins, petroleum, etc. Suitable co-solvents are for example t-BuOH, i-BuOH, 2-ethyl hexanol, 2-propyl heptanol, butyl glycols,

### D4) Dehazers

Dehazers/demulsifiers suitable for use in fuels are well known in the art as. As non-limiting examples there may be mentioned glycol oxyalkylate polyol blends (such as sold under the trade designation TOLAD™ 9312), phenol/formaldehyde or C₁₋₁₈ alkylphenol/-formaldehyde resin oxyalkylates modified by oxyalkylation with C₁₋₁₈ epoxides and diepoxides (such as sold under the trade designation TOLAD™ 9308), and C₁₋₄ epoxide copolymers cross-linked with diepoxides, diacids, diesters, diols, diacrylates, dimethacrylates or diisocyanates, and blends thereof. The glycol oxyalkylate polyol blends may be polyols oxyalkylated with C₁₋₄ epoxides. The C₁₋₁₈ alkylphenol phenol/formaldehyde resin oxyalkylates modified by oxyalkylation with C₁₋₁₈ epoxides and diepoxides may be based on, for example, cresol, t-butyl phenol, dodecyl phenol or dinonyl phenol, or a mixture of phenol (such as a mixture of t-butyl phenol and nonyl phenol). The dehazer should be used in an amount sufficient to inhibit the hazing that might otherwise occur when the fuel without the dehazer contacts water, and this amount will be referred to herein as a “haze-inhibiting amount.” Generally, this amount is from about 0.1 to about 10 ppm based on the weight of the fuel.

### D5) Further Coadditives

Further customary additives (different from those of the invention are) are corrosion inhibitors, for example based on ammonium salts of organic carboxylic acids, said salts tending to form films, or of heterocyclic aromatics for nonferrous metal corrosion protection; antioxidants or stabilizers, for example based on amines such as pphenylenediamine, dicyclohexylamine or derivatives thereof or of phenols such as 2,4-di-tert-butylphenol or 3,5-di-tert-butyl-4-hydroxyphenylpropionic acid; antistats; metallocenes such as ferrocene; methylcyclopentadienylmanganese tricarbonyl; lubricity additives, such as certain fatty acids, alkenylsuccinic esters, bis(hydroxyalkyl) fatty amines, hydroxyacetamides or castor oil; and also dyes (markers). Amines are also added, if appropriate, to lower the pH of the fuel. Optionally anti valve seat recession additives may be used such as sodium or potassium salts of polymeric organic acids.

### E) Additive Packages

The additives may be added to the fuel individually or as a concentrate (additive package) comprising a mixture of additives and solvents as discussed above.

Usually reaction products of the invention are blended with other fuel additives such as detergents, carrier oils, solvent, co-solvent, and other optional minor components as described above.

Typically, such packages may contain:
reaction product(s) of the invention: in proportions of about 5-80 or abot 10-70 or about 10-40 wt.-%, based on the total weight of the package;
detergent(s): in proportions of about 10-80 or about 20-70 or about 30-70 wt.-%, based on the total weight of the package;
carrier oil(s): in proportions of about 5-70 or about 10-50 or about 10-40 wt.-%, based on the total weight of the package;
solvent(s): in proportions of about 5-70 or about 5-50 or about 10-50 wt.-%, based on the total weight of the package;
Co-solvent(s): in proportions of about 1-40 or about 5-30 or about 5-20 wt.-%, based on the total weight of the package;
optionally: dehazer(s) (about <1%), corrosion inhibitor(s) (about 0, 1-5%), conductivity improvers (about <2%), each based on the total weight of the package; and others.

All components are blended to an additive package, which will be transported and stored for some days up to many months. In particular in cold regions or regions with cold winter season the package must therefore be stable for many weeks at deep temperatures. Stable means that no phase separation or precipitation occurs and the package must not become a solid stuff.

For example, the fuel additive package of the present invention remains a fluid at 0° C., or −8° C., or −18° C., or −20° C., or −30° C. or even −40° C. The fuel additive package in its fluid state is substantially free of precipitate and/or sediment. The fluid may be substantially free from suspended particles, flocculent, and substantial phase separation (i.e., no multiple phases are formed).

The reaction products of the invention are added to the fuel typically in an amount of from 5 to 2,000 ppm by weight, in particular from 10 to 1,500 or 10 to 500 ppm by weight. The other components and additives mentioned above are, if desired, added in amounts customary for the intended purpose.

### E) Fuels

The additive compositions according to the invention are useful in all conventional diesel and gasoline fuels, as described, for example, in Ullmann's Encyclopedia of Industrial Chemistry, 5th Ed. 1990, Volume A16, p. 719 ff.

For example, it is possible to use them in a gasoline fuel having an aromatics content of not more than 60% by volume, for example not more than 42% by volume or not more than 35% by volume, and/or a sulfur content of not more than 2000 ppm by weight, for example not more than 150 ppm by weight or not more than 10 ppm by weight.

The aromatics content of the gasoline fuel is, for example, from 10 to 50% by volume, for example from 30 to 42% by volume, in particular from 32 to 40% by volume or not more than 35% by volume. The sulfur content of the gasoline fuel is, for example, from 2 to 500 ppm by weight, for example from 5 to 100 ppm by weight, or not more than 10 ppm by weight.

In addition, the gasoline fuel may have, for example, an olefin content of up to 50% by volume, for example from 6 to 21% by volume, in particular from 7 to 18% by volume; a benzene content of up to 5% by volume, for example from 0.5 to 1.0% by volume, in particular from 0.6 to 0.9% by volume, and/or an oxygen content of up to 25% by volume, for example up to 10% by weight, or from 1.0 to 2.7% by weight, in particular from 1.2 to 2.0% by weight.

Examples of such gasoline fuels are in particular those which simultaneously have an aromatics content of not more than 38 or 35% by volume, an olefin content of not more than 21% by volume, a sulfur content of not more than 50 or 10 ppm by weight, a benzene content of not more than 1.0% by volume and an oxygen content of from 1.0 to 2.7% by weight.

The contents of alcohols and ethers in the gasoline fuel may vary over a wide range. Examples of typical maximum contents are 15% by volume for methanol, 65% by volume for ethanol, 20% by volume for isopropanol, 15% by volume for tert-butanol, 20% by volume for isobutanol and 30% by volume for ethers having 5 or more carbon atoms in the molecule.

The summer vapor pressure of the gasoline fuel is typically not more than 70 kPa, in particular 60 kPa (each at 37° C.).

The RON of the gasoline fuel is generally from 75 to 105. A typical range for the corresponding MON is from 65 to 95.

The specifications mentioned are determined by customary methods (DIN EN 228).

The invention will now be illustrated in detail with reference to the working examples, which follow:

### EXPERIMENTAL PART

### Example 1

### Reaction Product of Coconut Oil Methyl Ester and Diethanol Amine (Molar Ratio: 1:1)

A 5 L four-neck glass reactor equipped with condenser, automatic injection equipment, internal temperature control and anchor stirrer was charged with 2200 g of coconut methyl ester (technical grade: ester content, % (m/m): 96. 5 min, kinematic viscosity at 40° C., mm2/s: 2.0-4.5) and heated to 150° C. 1050 g of diethanol amine was added at this temperature within 30 minutes. The reaction mixture was kept at 150° C. for 4 hours, and than heated up for 1 hour to 160° C. to completely remove residual methanol. The resulting product was yellow oil.

### Example 2

### Reaction Product of Coconut Oil Methyl Ester and Diethanol Amine (Molar Ratio: 2:1)

According to procedure of example 1 3000 g of coconut methyl ester (technical grade: ester content, % (m/m): 96. 5 min, kinematic viscosity at 40° C., mm2/s: 2.0-4.5) and 716 g diethanol amine were reacted to a yellow oil.

### Example 3

### Reaction Product of Coconut Oil Methyl Ester and Diethanol Amine (Molar Ratio: 3:1)

According to procedure of example 1 3000 g of coconut methyl ester (technical grade: ester content, % (m/m): 96.5 min, kinematic viscosity at 40° C., mm2/s: 2.0-4.5) and 477 g diethanol amine were reacted to a yellow oil.

### Example 4

### Reaction Product of Oleic Acid and AEAE (Molar Ratio: 1:1)

A 250 ml glass flask equipped with a condenser was charged with 56.4 g of oleic acid (approx. 0.2 moles) and heated up to 130° C. At this temperature 20.8 g (0.2 moles) of amino ethyl ethanolamine were added within 10 minutes. After stirring for three hours at this temperature the reaction mixture was heated up to 180° C. and kept at this temperature for 5 hours. 66 g of brown oil was yielded which solidified after few hours to a light brown wax. Amine number was 124 mgKOH/g.

### Example 5

### Reaction Product of Oleic Acid and Aeae (Molar Ratio: 2:1)

Oleic acid and amino ethyl ethanolamine were reacted as described in Example 4 but in a molar ratio of 2:1. Resulting product was a light brown wax with an amine number of 14 mgKOH/g.

### Example 6

### Reaction Product of Oleic Acid and AEAE (Molar Ratio: 3:1)

Oleic acid and amino ethyl ethanolamine were reacted as described in Example 4 but in a molar ratio of 3:1. Resulting product was brown oil with an amine number of 6.2 mgKOH/g.

### Test Example 1

To demonstrate the effect of different molar ratios, three different products (prepared according to Example 4, 5 and 6) were blended with polyisobutene amine (PIBA), polyoxyalkylene carrier oil and different amounts of solvent to result in typical fuel additive compositions.

The storage stability at low temperature and the tendency to stabilize emulsions were examined. Standard test procedures were applied. The results are summarized in the following Table 1.

TABLE 1
Test results

| | | | | | | | | | | | Visual assessment |
|---|---|---|---|---|---|---|---|---|---|---|---|
| | | | | | | | | | | ASTM | after |
| | | | | Ex. 4 | Ex. 5 | Ex. 6 | | | ASTM | D 1094, | storage at |
| | Dose | | | 1:1¹⁾ | 2:1 | 3:1 | | | D 1094 | 5 min. + 1 ppm | −20° C. |
| | [mg/kg] | PIBA | PE | 1:3²⁾ | 2:3 | 3:3 | SNH | 2-PH | 5 min. | Dehazer³⁾ | for 7 days |
| mod1 | 1150 | 250 | 200 | 100 | | | 300 | 300 | 4/3⁴⁾ | 2/3 | Precipitation |
| mod2 | 1150 | 250 | 200 | | 100 | | 300 | 300 | 4/2 | 0/1 | Clear liquid |
| mod3 | 1150 | 250 | 200 | | | 100 | 300 | 300 | 2/1 | 0/1 | Clear liquid |
| mod4 | 1000 | 250 | 200 | 100 | | | 150 | 300 | | | Solid |
| mod5 | 1000 | 250 | 200 | | 100 | | 150 | 300 | | | Clear liquid |
| mod6 | 1000 | 250 | 200 | | | 100 | 150 | 300 | | | Clear liquid |
| mod7 | 850 | 250 | 200 | 100 | | | 150 | 150 | | | Solid |
| mod8 | 850 | 250 | 200 | | 100 | | 150 | 150 | | | Precipitation |
| mod9 | 850 | 250 | 200 | | | 100 | 150 | 150 | | | Turbidity |

¹⁾molar ratio of fatty acid and alkanol amide reactants
²⁾molar ratio of functional groups of fatty acid and alkanol amide reactants
³⁾Dehazer: commercial product containing oxalkylated polymers SNH = Solvent Naphta heavy 2-PH = 2-Propylheptanol
⁴⁾Rating scale according ASTM D 1094 interface/separation

This investigation clearly demonstrates that product of Example 6 requires less solubilizer to achieve stable formulations. At the same time products of Examples 5 and 6 are less critical in the ASTM D 1094 test.

While the present invention was exemplified by making reference to the reaction products obtained with two specific reactants, a skilled reader will be enabled, guided by the general teaching as provided herein, to perform the invention without undue burden with other reactants of the general formulae I and II in order to prepare superior fuel additives falling within the scope of the present invention.

The disclosure of the citer prior art is incorporated by reference.

1. A reaction product, obtainable by reacting a carboxylic acid compound of formula I
R¹COOR² (I)
in which
R¹ is an aliphatic C₁-C₃₀-hydrocarbon radical;
R² is hydrogen or alkyl, mono- or polyhydroxyalkyl, or ammonium,
with an alkanol amine of the formula II
NHR³R⁴ (II)
wherein R³ and R⁴ are independently selected from hydrogen atoms and linear or branched-chain hydrocarbon groups, the carbon chain of which optionally being interrupted by one or more —NH— groups, and which optionally has at least one hydroxyl group attached to a carbon atom, with the proviso that R³ and R⁴ are not both hydrogen atoms and that at least one of said residues R³ and R⁴ carries at least one hydroxyalkyl group,
in a molar ratio of the carboxyl groups of the carboxylic acid of formula I to the molar sum of OH and NH groups of the alkanol amine of formula II in a range and under reaction conditions supporting the formation of a reaction product comprising polysubstituted alkanol amine derivatives.

2. The reaction product of claim 1, comprising said polysubstituted alkanol amine derivatives in a proportion of more than 20 wt.-%, preferably more than 40 wt.-%, and in particular more than 60 wt.-%, based on the total weight of the product.

3. The reaction product of claim 1 or 2, wherein the molar ratio of the carboxyl groups of the carboxylic acid of formula Ito the molar sum of OH and NH groups of the alkanol amine of formula II is in the range of about 1.8:3 to 3:3, in particular 1.9:3 to 2.5:3.

4. The reaction product of anyone of claims 1 to 3, wherein the reaction is performed by
a) heating the carboxylic acid of formula I to a first temperature in a first temperature range, allowing the preferential reaction of the acid with amine group(s) of the alkanol amine;
b) adding thereto the alkanol amine of formula II under controlled conditions in order to avoid an increase of the temperature above said first temperature range;
c) reacting the compounds by maintaining the temperature in said first range;
d) and increasing the first temperature of the reaction mixture to a second temperature in a second temperature range allowing further condensation of residual free carboxylic acid molecules with any reactive group in the reaction mixture.

5. The reaction product of claim 4, wherein the first temperature in step a), b) and/or c) is kept in the range of 100 to 155° C.

6. The reaction product of any one of the claims 4 and 5, wherein the second temperature in step d) is kept in the range of 160 to 210° C.

7. The reaction product of any one of the preceding claims, wherein R³ and R⁴ independently of each other represent hydrogen or a residue of the formula III
—[(CH₂)ₓNH]_{y}(CH₂)_{z}R⁵ (III)
wherein
x and z are independently from each other integers from 1 to 6,
y is 0 or an integer of 1 to 3 and
R⁵ is hydroxyl or a residue of the formula IV
—NH(CH₂)_{z}OH (IV)
wherein z is as defined above; with the proviso that R³ and R⁴ are not both hydrogen atoms.

8. The reaction product of any one of the preceding claims, wherein the compound of formula I is selected from C₈-C₃₀-carboxylic acids and alkyl esters thereof.

9. The reaction product of any one of the preceding claims, wherein the compound of formula II is selected from polyaminoalkanols, wherein one of the residues R³ and R⁴ is hydrogen and the other is a residue of the formula III, wherein x is 2 or 3, y is 0 or 1, z is 2 or 3 and R⁵ is hydroxyl or a residue of the formula IV.

10. An additive package, comprising in a suitable organic solvent at least one detergent additive and at least one reaction product of anyone of the claims 1 to 9.

11. A method of improving the storage stability of additive packages, wherein the additive package comprises at least one detergent additive in an organic solvent, which method comprises adding to said package at least one reaction product of one of the claims 1 to 9

12. The method of claim 11, wherein the detergent additive is selected from as polyalkene monoamines, polyalkene Mannich amines or polyalkene succinimides.
